(19) United States
(12) Patent Application Publication
(10) Pub. No.: US 2022/0213429 A1
(43) Pub. Date: Jul. 7, 2022

(54) METHOD AND MEANS FOR OPTIMIZING BIOTECHNOLOGICAL PRODUCTION

(71) Applicant: INSILICO BIOTECHNOLOGY AG, Stuttgart (DE)

(72) Inventors: Bastian NIEBEL, München (DE); Klaus MAUCH, Stuttgart (DE); Joachim SCHMID, Sindelfingen (DE); Matthias BOHNER, Stuttgart (DE)

(21) Appl. No.: 17/609,204

(22) PCT Filed: May 8, 2019
(86) PCT No.: PCT/EP2019/061878

§ 371 Date: Nov. 5, 2021

(51) Int. Cl. C12M 1/36; G05B 13/02; G06N 3/04

(52) U.S. Cl. CPC C12M 41/48; G06N 3/0445; G06N 3/0481; G05B 13/027

(57) ABSTRACT

A new method for the automatic generation and validation of a Digital Twin for the production of biotechnological products and the application of the Digital Twin for the purpose of increasing product concentration, productivity, biomass concentration and product quality by optimizing media composition and/or feeding profiles. The Digital Twin can be linked directly to production for online optimization or offline for decision support.

The invention provides a new method for the automatic generation and validation of a Digital Twin for the production of biotechnological products and the application of the Digital Twin for the purpose of increasing product concentration, productivity, biomass concentration and product quality by optimizing media composition and/or feeding profiles. The Digital Twin can be linked directly to production for online optimization or offline for decision support.

Today, Digital Twins are used in mechanical engineering, electrical engineering, in the chemical industry and other related industries as they may significantly improve and speed-up design, optimization and control of machines, industrial products, and supply chains. Through their predictive qualities, Digital Twins can be used to intervene directly in production or to predict and improve the overall behavior of assets and the supply chain. Despite such advantages, Digital Twins are not applied in biotechnological production processes.

Although models of biotechnological processes have been developed in the past, most of these models cannot tackle three main issues: (i) the models have hardly any predictive qualities, (ii) necessary experimental data cannot be provided as required, (iii) the creation of the models is too expensive due to the complexity of the cellular system or impossible due to too many unknowns.

### SUMMARY

The inventors have found, for the first time, methods and means for a highly predictive Digital Twin by combining a cell model, a reactor model, a growth model and extracellular reaction kinetics with machine learning (see FIG. 1). Through a quasi-stationary description of intracellular concentrations, the Digital Twin can be trained and validated solely on the basis of the dynamics of substrates, products (i.e. “compounds”) and biomass. These experimental data can be easily provided on a routine bases and are standard measurement data in most biopharmaceutical production processes. The invention capitalizes on a) the mechanisms of well-known metabolic networks as well as the well-described cultivation systems and b) the data-driven learning of unknown cellular mechanisms through machine learning.

Training, validation and application of the Digital Twin is fully automated, interchangeable between different process formats like continuous, batch and fed-batch cultivations, interchangeable between different products like monoclonal antibodies, antibody fragments, vitamins, amino acids, hormones or growth factors. In addition, the method can be applied to all organisms and cell lines for which metabolic networks have either been reconstructed or can be reconstructed.

### SUMMARY OF THE FIGURES

FIG. 1 shows a schematic representation of the structure of the Digital Twin according to the invention.

FIG. 2 schematically shows the function of the Digital Twin and its applications in real cell culture systems.

FIG. 3 is a flow chart of an implemented workflow for a method according to a preferred embodiment of the invention

FIG. 4 is a flow chart of a phase and exchange rate estimation algorithm.

FIG. 5 is a flow chart of a metabolic flux analysis algorithm.

FIG. 6 is a flow chart for deriving elementary flux modes.

FIGS. 7A and 7B show schematic representations of a recurrent metabolic network model.

FIG. 8 is a schematic representation of the matrix multiplication algorithm according to the invention.

FIG. 9 is a flow chart of a training and evaluation algorithm for a recurrent metabolic network model.

FIG. 10 is a flow chart of a process optimization algorithm.

FIG. 11 is a flow chart of a complete workflow of a method according to the invention.

FIG. 12 shows graphs on the performance of a Digital Twin according to the invention.

FIGS. 13 and 14 show graphs on measured and predicted concentration of biomass and product.

### DETAILED DESCRIPTION OF THE INVENTION

There is provided a Digital Twin for a cell cultivation process, the Digital Twin represents a plurality of a biological cell, extracellular reactions and a reactor system.

In a first aspect, the invention provides a method for the construction of the Digital Twin:
providing dynamic cultivation data from a real cell cultivation process;
providing a mode matrix M of elementary flux modes, extracted from metabolic fluxes of a real biological cell;
reducing the number of and overlaying the elementary flux modes by a trainable matrix H to obtain a reduced matrix \tilde{S}\tilde{M}^{red} of base flux modes;
assigning a neural network for describing the kinetics of the individual base flux modes \tilde{S}\tilde{M}^{red};
connecting the base flux modes to extracellular reactions of the cell cultivation process;
connecting the base flux modes to inflows and outflows to and from the reactor system of the cell cultivation process;
solving the resulting mass balances of substrates, products and biomass; and
training the H matrix and the neural network by the dynamic cultivation data.

The H matrix is a unique feature of this embodiment of the present invention. According to the invention, H is a trainable matrix with two functions:

(i) it transforms the number of elementary flux modes Num^{modes} to a reduced number Num^{modes,red} (i.e. dimensionality reduction)

(ii) it combines the modes through a matrix multiplication operation (i.e. mode combination).

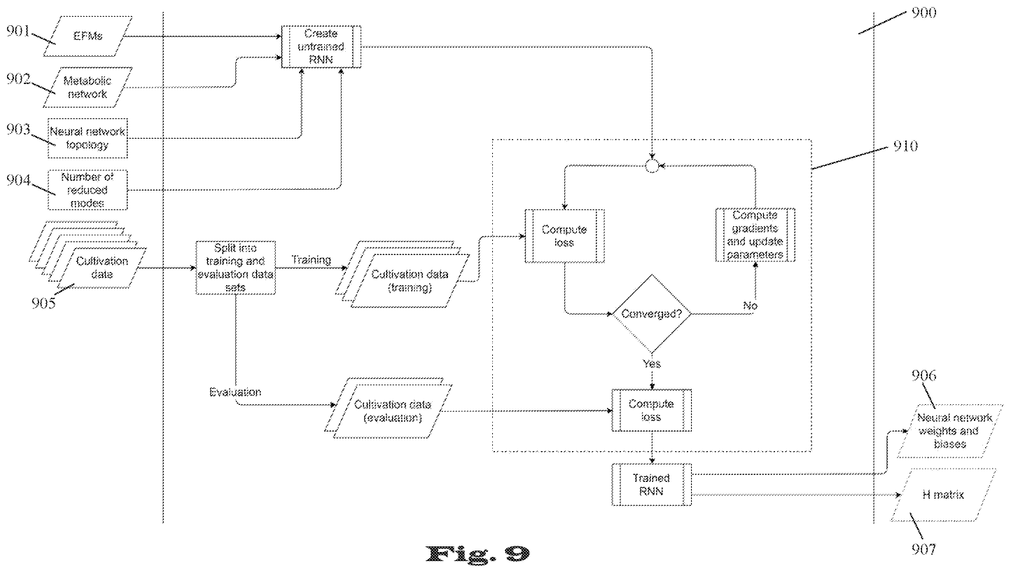

The matrix multiplication according to the present invention thus leads to a projected reduced stoichiometric matrix \tilde{S}\tilde{M}^{red} with its rows corresponding to the number of reduced modes Num^{modes,red} and its columns corresponding to the number of measured compounds Num^{comp,measured}. FIG. 8 depicts a schematic representation of the matrix multiplication operation which reduces the mode dimensionality.

The projected reduced stoichiometric matrix \tilde{S}\tilde{M}^{red} is preferably derived from metabolic network matrices \tilde{S} and \tilde{M} by applying the trainable positive reduction matrix H to transform the number of modes Num^{modes} to a reduced number Num^{modes,red}:

\tilde{S}\tilde{M}^{red}=H·[\tilde{S}·\tilde{M}^{T}]^{T}

h_{u,z}≥0∧h_{u,z}∈H

wherein in particular the metabolic network matrices \tilde{S} and \tilde{M} are derived from a stoichiometric matrix S of the real biological cell and said flux mode matrix M, by removing all exchange reactions from both matrices, and wherein in \tilde{S} only the exchange compounds are included.

The method of the invention requires a solver for the mass balances of substrates, products and biomass. In a preferred embodiment, the solver is a recurrent neural network (RNN). This RNN preferably comprises the following components:
an intermediate state model, to describe the changes in the cultivation volume and the state vector as a continuous function of time for a certain time step t while ensuring correct mass balance;
said neural network, to compute the update of the base flux modes f(t) by training the neural network weights W along with their corresponding biases b, where the neurons of the next layer are activated by a sigmoidal activation function σ:

f(t)=σ(W_{L}·σ(W_{L-1}· . . . ·σ(W₀·\tilde{X}(t)+b₀)+ . . . +b_{L-1})+b_{L})

wherein L denotes the index of the last hidden layer;
a flux-based rate estimation to obtain the extracellular rates by:

[μ(t), r(t)]=f(t)·\tilde{S}\tilde{M}^{red}=f(t)·H·[\tilde{S}·\tilde{M}^{T}]^{T}

h_{u,z}≥0∧h_{u,z}∈H

and
an exponential growth model to calculate the state vector for the next time step t+Δt

In a preferred variant thereof, the training of the RNN is performed by using a first subset of the cultivation data, the so called training set, by minimizing Loss in the following loss function:

$$Loss = {\sum }_{p , t , i} {( \frac{{c}_{p , i}^{predicted} ( t ) - {c}_{p , i}^{m e a s u r e m e n t} ( t )}{{c}_{p , i}^{measurement , std} ( t )} )}^{2}$$

where i is an indication of compounds including biomass, c_{p,i}^{measurement}(t) is the measured concentration of compound i, c_{p,i}^{measurement,std}(t) is the measurement standard deviation of the concentration of compound i, c_{p,i}^{predicted}(t) is the predicted concentration of compound i, each at time point t and each corresponding to the selected cultivation run p.

In a preferred variant thereof, the evaluation of the trained RNN is performed by calculating said Loss on the basis of a second subset of the cultivation data, so called evaluation set, the second subset (evaluation set) being different from the first subset (training set) of data used for training.

In a particular embodiment of the method of the invention, the mode matrix M of the elementary flux modes is obtained by a method of mode decomposition. Preferably, the method comprises the steps of:
transforming the set of all metabolic fluxes to separate off reversible reactions to obtain a set of all irreversible reactions;
minimizing an objective function and deactivation of inactivate transformers recurrently applied to obtain elementary flux modes, the objective function being:

min(Num^{rxns,v}^{_{nonzero}}⁾
where Num^{rxns,v}^{_{nonzero}} is the number of reactions with non-zero fluxes; and
collecting all identified elementary flux modes and stacking them into a mode matrix M.

According to this aspect, the invention provides a Digital Twin representing (i) a reactor model, an extracellular reaction model, and the cell model, (ii) a machine learning step (i.e. a neural network), and (iii) a process optimization step applied to the real biological system.

The reactor model includes all the in- and outlets to/from the cultivation system, including but not being limited to feeding, sampling (and compensation), cell bleeding, and permeate outflow. The reactor model thus describes the exchange of liquid and gas along with the associated exchange of substrates, products and biomass to/from the cultivation system.

The extracellular reaction model includes all chemical reactions taking place in the cultivation media, including but not being limited to degradation processes such as the oxidation of metabolites like glutamine or the fragmentation of products like antibodies.

The cell model includes all known metabolic pathways including transport steps, such as glycolysis, amino acid metabolism, amino acid degradation, the formation of DNA/RNA, protein, lipids, carbohydrates, glycosylation, respiration and transport steps between intracellular compartments as well as between the cytosol and the extracellular environment. For the calculation of elementary flux modes, the elemental and charge balance of the individual stoichiometric reactions and transport steps in the cell model must be ensured.

The machine learning step comprises the neural network f(t) which receives the real (i.e. experimental) cultivation data as inputs for training. This trained neural network, in turn, predicts the fluxes of the base modes including consumption and production rates of all compounds including biomass involved in the process at each time point based on the process state of the previous time point.

In a preferred embodiment, the said Digital Twin is formulated in a matrix format as:

$$\begin{matrix}\frac{d}{d t} X ( t ) = ( G ( t ) + A + D ( t ) ) * X ( t ) + F ( t ) * {X}^{I} ( t ) \\ = E ( t ) * X ( t ) + F ( t ) * {X}^{I} ( t )\end{matrix}$$

where X(t) denotes the state vector (vector of all concentrations), G (t) comprises the growth terms for every compound (representing the cell model), A represents the extracellular model (here the glutamine degradation), D(t) comprises the outflow rates (i.e. sampling, cell bleeding, and permeate), F(t) comprises the inflow rates (i.e. sampling inflow and volumetric feed) and X^{I}(t) comprises the feed-concentrations of all compounds in the media, and E(t) (as the sum of G(t), A, and D(t)) is the system matrix. F(t) together with D(t) represents the reactor model.

The matrices according to a preferred embodiment of the invention are described in more detail follows:

$$X ( t ) = [ \begin{matrix}x ( t ) \\ {c}_{glu} ( t ) \\ {c}_{amn} ( t ) \\ {c}_{5 - ox} ( t ) \\ {c}_{i} ( t )\end{matrix} ] \\ G ( t ) = [ \begin{matrix}μ ( t ) & 0 & 0 & 0 & 0 \\ {r}_{glu} ( t ) & 0 & 0 & 0 & 0 \\ {r}_{amn} ( t ) & 0 & 0 & 0 & 0 \\ {c}_{5 - ox} ( t ) & 0 & 0 & 0 & 0 \\ {r}_{i} ( t ) & 0 & 0 & 0 & 0\end{matrix} ] \\ A = [ \begin{matrix}0 & 0 & 0 & 0 & 0 \\ 0 & 0 & - {k}^{deg} & 0 & 0 \\ 0 & 0 & {k}^{deg} & 0 & 0 \\ 0 & 0 & {k}^{deg} & 0 & 0 \\ 0 & 0 & 0 & 0 & 0\end{matrix} ] \\ D ( t ) = [ \begin{matrix}{k}_{1} ( t ) & 0 & 0 & 0 & 0 \\ 0 & {k}_{3} ( t ) & 0 & 0 & 0 \\ 0 & 0 & {k}_{3} ( t ) & 0 & 0 \\ 0 & 0 & 0 & {k}_{3} ( t ) & 0 \\ 0 & 0 & 0 & 0 & {k}_{2} ( t )\end{matrix} ] \\ F ( t ) = [ \begin{matrix}0 & 0 & 0 & 0 & 0 \\ 0 & {k}_{2} ( t ) & 0 & 0 & 0 \\ 0 & 0 & {k}_{2} ( t ) & 0 & 0 \\ 0 & 0 & 0 & {k}_{2} ( t ) & 0 \\ 0 & 0 & 0 & 0 & {k}_{2} ( t )\end{matrix} ] \\ {X}^{I} ( t ) = [ \begin{matrix}x ( t ) \\ {c}_{glu}^{I} ( t ) \\ {c}_{amn}^{I} ( t ) \\ {c}_{5 - ox}^{I} ( t ) \\ {c}_{i}^{I} ( t )\end{matrix} ] and \\ {k}_{1} ( t ) = - \frac{{F}_{B} ( t ) + {F}_{s}^{O} ( t )}{V ( t )} \\ {k}_{2} ( t ) = \frac{{F}_{F}^{I} ( t ) + {F}_{s}^{I} ( t )}{V ( t )} \\ {k}_{3} ( t ) = - \frac{{F}_{F}^{O} ( t ) + {F}_{B} ( t ) + {F}_{s}^{O} ( t )}{V ( t )} \qquad$$

where cᵢ(t) is the concentration of compound i (all compounds except Glutamine, Ammonia, and 5-Oxoproline which are represented by c_{glu}(t), cₐₘₙ(t), and c₅₋ₒₓ(t), respectively). x(t) and μ(t) are the biomass concentration and the exponential growth rate, respectively. rᵢ(t) is the reaction rate of the compound i (all compounds except Glutamine, Ammonia, and 5-Oxoproline which are represented by r_{glu}(t), rₐₘₙ(ₜ), and r₅₋ₒₓ(t), respectively). k^{deg} is the rate constant of abiotic degradation of Glutamine to Ammonia and 5-Oxoproline. V(t) is the culture volume. F_{B}(t), F_{S}^{O}(t), F_{S}^{I}(t), and F_{F}^{O}(t), are volumetric cell bleeding rate, non-continuous volumetric outflow rate e.g. sampling rate, volumetric feed inflow rate, non-continuous volumetric inflow rate e.g. sampling compensation rate, and permeate outflow rate, respectively.

In a preferred embodiment of the invention, the neural network structure is set up based on the neural network f(t) hyper-parameters. Hyper-parameters of the neural network may include, but are not limited to: generalization parameters (batch size and dropout rate), learning rate, the optimizer type, and the topology of the neural network (i.e. number of hidden layers and number of neurons per layer).

In a preferred embodiment of the invention, a pre-processing of the cultivation data is performed. A preferred variant the pre-processing of the cultivation data includes the steps of (i) quantization: mapping the time points of the actual measurement to the data sampling period, (ii) unit conversion: converting the units of all data to reach consistency, and (iii) compensation of missing data, aiming to fill missing data points, in particular by interpolation.

In preferred embodiments of the invention, the Digital Twin can be constructed in a method employing three consecutive steps: Flux Analysis, Mode Decomposition, and Training/Validation by the Recurrent Neural Network (RNN).

In the following, the steps of flux analysis according to a preferred embodiment of the invention are described in more detail: To quantify the cellular fluxes, flux analysis is performed in two consecutive steps: (i) phase search and exchange rate estimation, followed by (ii) Metabolic Flux Analysis (MFA).

In a preferred embodiment of the invention, phase search and exchange rate estimation in flux analysis are as follows: The molar amounts of all compounds of interest in the system including biomass are computed by

N(t+Δt)=X (t+Δt)·V (t+Δt)

where

$$X ( t + Δ t ) = {\sum }_{i = 1}^{m} {q}_{i} v e {c}_{i} {e}^{{val}_{i} Δ t} + {\sum }_{i = 1}^{m} {Q}_{i} ( Δ t ) {e}^{{val}_{i} Δ t}$$

with m being the number of compounds in the system including biomass. νalᵢ and νecᵢ are the eigenvalues and eigenvectors of \tilde{E}, respectively. qᵢ is a constant value depending on the starting conditions (at time t). Qᵢ(Δt) is calculated by variation of constants and represents the particular solution of the process equation.

Biomass growth is divided into different phases considering a quasi-steady state within each phase. This means that the growth rate, the biomass-specific fluxes and exchange rates are considered to be constant.

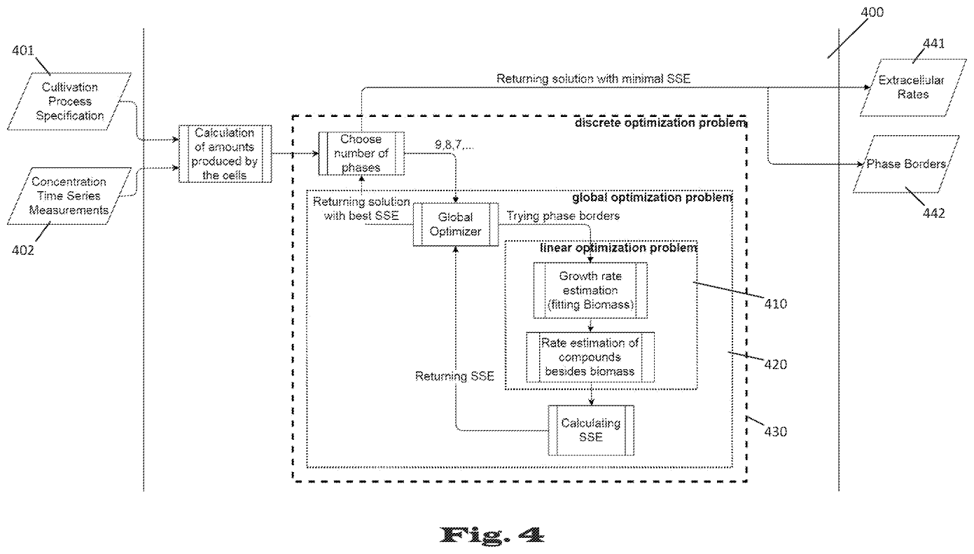

According to a preferred aspect thereof, the overall procedure of phase search is a three times nested optimization algorithm (FIG. 4). This preferred aspect of the present invention provides:
A linear convex problem, which is about estimating growth rate of biomass and the rates of all compounds besides biomass, corresponding to each estimated phase.
A global continuous problem, that finds the optimized positions of the phase borders, it has a local minimum, and a global optimizer can be used to solve it.
A discrete optimization problem, which defines the best number of phases by minimizing the Sum of Squared Error (SSE) between the estimated and the measured amounts.

To summarize, the solution of the linear convex problem provides the exchange rates, the global continuous problem finds the optimized positions of the phase borders, and the discrete optimization problem estimates the best number of phases. Inputs to the phase search and exchange rate estimation are cultivation data and the outputs are the estimated extracellular rates (i.e. exchange rates) and the estimated phase borders. By applying phase search and exchange rate estimation, the cellular exchange rates of all compounds corresponding to each estimated phase are quantified.

In the following, Metabolic Flux Analysis (MFA) according to a preferred embodiment of the invention is described in more detail: Given the estimated exchange rates of all compounds within each phase, the next step of this preferred embodiment of the present invention is to quantify the intracellular rates corresponding to each phase.

The intracellular fluxes are computed based on the work of Antoniewicz et al. [1], with the substantial difference that according to this preferred embodiment of the invention the objective function is a Weighted Mean Squared Error (WMSE) and a penalty factor controlling the complexity is added to the objective function:

$${min}_{{v}_{j k} , {b}_{j}} λ \cdot {\sum }_{j ∈ r x n {s}^{e x c h a n g e}} ( {\sum }_{k ∈ conditions} {( \frac{{v}_{j k}^{e s t} - {r}_{j k}^{e s t}}{{r}_{j k}^{std}} )}^{2} ) + ( 1 - λ ) \cdot {\sum }_{j ∈ r x n s} {b}_{j}$$

Subject to:

$${\sum }_{j ∈ r x n s} {S}_{i j} \cdot {v}_{j k} = 0 ∀ i ∉ m e t {s}^{m e asured} ⩓ ∀ k ∈ conditions$$

$${v}_{j k} \geq 0 ∀ j ∈ irreversible rxns ⩓ ∀ k ∈ conditions$$

$${b}_{j} = 0 ⇒ {v}_{j k} = 0 ∀ j ∈ r x n s ⩓ ∀ k ∈ conditions$$

where νⱼₖ^{est} indicates the estimated exchange rates from MFA corresponding to reaction j and condition k. rⱼₖ^{est} and rⱼₖ^{std} indicate the mean and the standard deviation of the estimated exchange rates, respectively, from phase search and exchange rate estimation corresponding to reaction j and condition k. The condition k stands for each phase of a cultivation process. b₁ is a binary variable corresponding to reaction j representing the complexity which is defined as:

$${b}_{j} = \{ \begin{matrix}0 , & if reaction j is excluded in the model \\ 1 , & if reaction j is excluded in the model\end{matrix}$$

wherein any flux νⱼ corresponding to bⱼ=0 is set to 0:

bⱼ=0⇒νⱼ=0∀j∈rxns

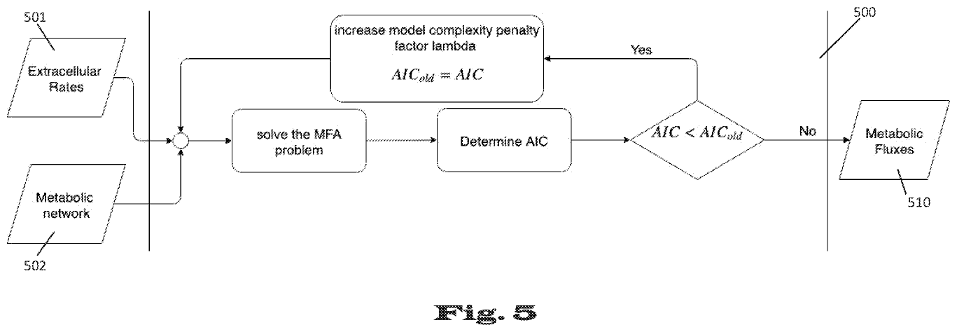

λ is a penalty factor, which ranges between 0 and 1, weighting the model complexity Σ_{j∈rxns}bⱼ against the estimated fluxes. Sᵢⱼ is an element of the stoichiometric matrix of the metabolic network corresponding to metabolite i and reaction j. According to this preferred embodiment of the invention, Akaike Information Criterion (AIC) is employed for a series of λ values to select the best model (see FIG. 5). The inputs to the MFA algorithm are the estimated extracellular rates (obtained from the phase search and exchange rate estimation), and the process model. The output of the MFA is a set of intracellular metabolic fluxes.

According to the invention, products such as therapeutic proteins can be formed from monomers like amino acids. To derive the stoichiometric factors of product formation in an automated way from a different product composition, the following calculation is carried out:

$${s}_{m o {n}_{i}} = \frac{C h L}{{\sum }_{i} {f}_{m o {n}_{i}}} \cdot {f}_{m o {n}_{i}}$$

where ChL is the average chain length (i.e. the average amount of amino acids combined in a chain of the product). fₘₒₙ_{ᵢ} indicates the monomer factor of the i^{th} monomer monₛ and sₘₒₙ_{ᵢ} denotes the stoichiometric coefficient of the i^{th} monomer in the product protein synthesis reaction.

In addition, and according to a preferred embodiment of the invention, the energy consumption for protein chain elongation is considered. Each individual elongation step is performed at the cost of the equivalents to 4 ATP molecules that are hydrolysed to ADP and inorganic phosphate, Pᵢ. The corresponding partial reaction ATP+H₂O→ADP+Pᵢ also represents other equivalent energy providing hydrolysis reactions such as GTP+H₂O→GDP+Pᵢ or 0.5 ATP+H₂O→0.5 AMP+Pᵢ.

To produce a protein of length ChL we need ChL−1 binding reactions, where the peptide bond formation provides the water needed for ATP hydrolysis. Accordingly, the whole stoichiometric equation for product protein formation is as follows:

$${\sum }_{i} {s}_{m o {n}_{i}} m o {n}_{i} + 4 \cdot ( Ch L - 1 ) \cdot ATP = Product + 4 \cdot ( Ch L - 1 ) \cdot {P}_{i} + 4 \cdot ( Ch L - 1 ) \cdot ADP$$

In an alternative implementation, the invention also includes the formation of products that include other constituents than amino acids, such as glycosyl residues.

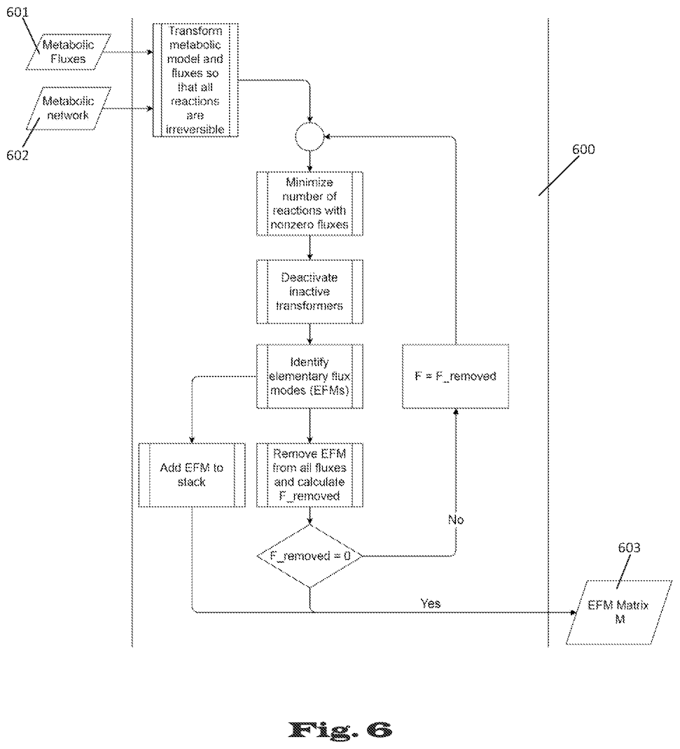

In the following, the steps of Mode Decomposition to obtain the elementary flux modes according to a preferred embodiment of the invention is described. Computing the complete set of elementary flux modes in a standard way is computationally expensive and leads to a combinatorial explosion in genome-scale metabolic networks. To derive elementary flux modes according to a preferred aspect of the invention, the method proposed by Chan et al. [2] (cf FIG. 6) is applied with the modification in the objective function:

min(Num^{rxns,v}^{_{nonzero}}⁾

where Num^{rxns,v}^{_{nonzero}} is the number of reactions with nonzero fluxes. This modification minimizes the number of used reactions, leading to elementary flux modes with minimum number of reactions. The elementary flux modes can then be used in the form of mode matrix M as an input to train the Recurrent Neural Network (RNN) of the present invention.

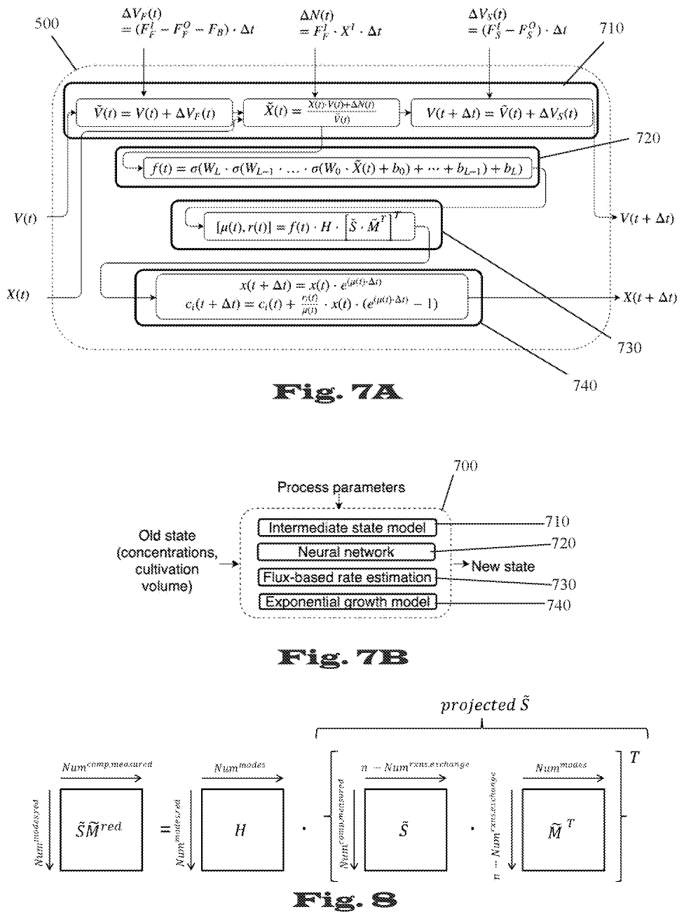

In the following, the Recurrent Neural Network (RNN) according to a preferred embodiment of the invention is described in more detail. The RNN consists of the intermediate state model, the neural network f(t), the flux-based rate estimation, and the exponential growth model (FIG. 7). The RNN is used to simulate feeding, metabolism and growth of the cell. The intermediate state model updates the cultivation volume and computes the intermediate state vector which is the input to the neural network f(t). The neural network f(t), in turn, then updates the base flux modes. The updated base flux modes are projected back onto the reduced stoichiometric matrix to get the exchange rates between cells and their environment for the next time step. The exponential growth model, in turn, is then used to update the state vector for the next time step based on the extracellular rates from the metabolic network (FIG. 7).

In the following, the intermediate state model of RNN according to a preferred embodiment of the invention is described. The intermediate state model describes the changes in the cultivation volume V(t) and the state vector \tilde{X}(t) (i.e. concentrations) as a continuous function of time for a certain time step while ensuring correct mass balance. Since the cultivation process also includes feeding media and sampling from the fermenter at specific time points, these discrete processes need to be taken into account separately. This is done in three distinct steps:

(i) Calculation of the intermediate volume \tilde{V}(t) by taking the continuous (i.e. feeding-related) cultivation volume change ΔV_{F}(t) into account:

$$\tilde{V} ( t ) = V ( t ) + {\overbrace{( {F}_{F}^{I} - {F}_{F}^{O} - {F}_{B} ) \cdot Δ t}}^{Δ {V}_{F} ( t )}$$

where V(t) is the cultivation volume, F_{F}^{I}, F_{F}^{O}, and F_{B}, are volumetric feed inflow rate, permeate outflow rate, and volumetric cell bleeding rate, respectively. Δt is the duration of a time step.

(ii) Calculation of the intermediate state vector \tilde{X}(t) based on the molar concentration formula:

$$\tilde{X} ( t ) = \frac{X ( t ) \cdot V ( t ) + {\overbrace{{F}_{F}^{I} \cdot {X}^{I} \cdot Δ t}}^{Δ N ( t )}}{\tilde{V} ( t )}$$

where X(t) is the state vector, X^{I} is the feed-concentration vector, and ΔN(t) is the amount change of the compounds due to feeding. The intermediate state vector \tilde{X}(t) is then used as input to the neural network.

(iii) Calculation of the cultivation volume V(t+Δt) by taking the non-continuous (i.e. sampling-related) cultivation volume change ΔVₛ(t) into account:

$$V ( t + Δ t ) = \tilde{V} ( t ) + {\overbrace{( {F}_{S}^{I} - {F}_{S}^{O} ) \cdot Δ t}}^{Δ {V}_{s} ( t )}$$

where F_{S}^{O} and F_{S}^{I} are non-continuous volumetric outflow rate and non-continuous volumetric inflow rate, respectively.

In the following, the flux based rate estimation of the invention as employed in the RNN according to a preferred embodiment is described in more detail: \tilde{S} and \tilde{M} are derived from the stoichiometric matrix S and the mode matrix M, by removing the columns corresponding to the exchange reactions in both matrices (i.e. number of columns=n−Num^{rxns,exchange} where n is the total number of reactions; see FIG. 8). In \tilde{S} only the exchange (i.e. measured) compounds are included (i.e. number of rows=Num^{comp,measured}). \tilde{S} and \tilde{M} are used to compute the projected S (see FIG. 8). According to this preferred embodiment of the present invention, the projected \tilde{S} along with a positive reduction matrix H (dimension: Num^{modes,red}×Num^{modes}) is used to compute the projected reduced stoichiometric matrix \tilde{S}\tilde{M}^{red}.

\tilde{S}\tilde{M}^{red}=H·[\tilde{S}·\tilde{M}^{T}]^{T}

h_{u,z}≥0∧h_{u,z}∈H

The matrix multiplication leads to a projected reduced stoichiometric matrix \tilde{S}\tilde{M}^{red} with its rows corresponding to the number of reduced modes Num^{modes,red} and its columns corresponding to the number of measured compounds Num^{comp,measured}.

In the next time step of the RNN, the growth rate μ(t) and extracellular rates r(t) are obtained by:

[μ(t), r(t)]=f(t)·\tilde{S}\tilde{M}^{red}=f(t)·H·[\tilde{S}·\tilde{M}^{T}]^{T}

h_{u,z}≥0∧h_{u,z}∈H

In the following, the exponential growth model of the RNN according to a preferred embodiment of the invention is described. The biomass concentration x(t+Δt) and compound concentrations cᵢ(t+Δt) for the next time step are calculated using analytical solution of the process model equation (see FIG. 7):

$$x ( t + Δ t ) = x ( t ) \cdot {exp}^{( μ ( t ) Δ t )}$$

$${c}_{i} ( t + Δ t ) = {c}_{i} ( t ) + \frac{{r}_{i} ( t )}{μ ( t )} \cdot x ( t ) \cdot ( {exp}^{( μ ( t ) \cdot Δ t )} - 1 )$$

where μ(t) and rᵢ(t) are the growth rate and the exchange rate of the iₜₕ compound at time point t.

In the following, the training and evaluation or verification of the RNN according to a preferred embodiment of the invention is described in more detail. During the training of the RNN, neural network weights W, biases b, and the H matrix are trained on the basis of a training set of the cultivation data, i.e. a subset of several cultivation runs. The neural network represents the kinetics of the cell and the H matrix is a mode reduction/combination matrix.

According to this preferred embodiment of the invention, the k-fold cross-validation method is applied to prevent over-fitting and to achieve a good generalization of the model. The gradients are updated by minimizing the following loss function:

$$Loss = {\sum }_{p , t , i} {( \frac{{c}_{p , i}^{predicted} ( t ) - {c}_{p , i}^{m e a s u r e m e n t} ( t )}{{c}_{p , i}^{measurement , std} ( t )} )}^{2}$$

where i is an indication of compounds including biomass, c_{p,i}^{measurement}(t) is the measured concentration of compound i, c_{p,i}^{measurement,std}(t) is the measurement standard deviation of the concentration of compound i, c_{p,i}^{predicted}(t) is the predicted concentration of compound i, each at time point t and each corresponding to the selected cultivation run p. The optimization problem is solved using an optimization algorithm i.e. stochastic gradient descent. Training is performed until the objective function converges to a value that does not significantly change anymore over a certain number of iterations (see FIG. 9). After a successful training, the RNN returns the trained H matrix and the learned weights and biases of the neural network.

In a preferred embodiment of the present invention, after successful training of the RNN using the training set of the cultivation data, the performance of the trained RNN is evaluated using the evaluation set of the cultivation data which is different from the training set (see FIG. 9). The performance of the trained RNN is evaluated using R² measure between the measured and the predicted concentrations of the cultivation process compounds. Other performance measurements can be used alternatively or additionally to evaluate the performance of the trained RNN.

The model uses hyper-parameters. In a preferred embodiment of the invention, a grid search is used to automatically find the optimum values for the hyper-parameters leading to the highest predictive power of the model (i.e. based on the best R² measure, see above). In a preferred embodiment of the invention, given the best hyper-parameter set, the model is re-trained with the complete training set.

After having finished the training and the evaluation of the RNN, the Digital Twin is can be readily used to optimize the process.

In a second aspect, the present invention provides a method for employing this Digital Twin to optimize the process specifications of a real biotechnological process to achieve a specific process optimization objective. The process specifications are particularly selected from, but not limited to the composition of the feed media and the feeding strategy. The process optimization objective is particularly selected from, but not limited to: maximization of product concentration, productivity, improvement of product quality, and maximization of biomass concentration within given process optimization constraints, such as fermenter volume, feeding amounts, feeding time points, and compound concentrations.

According to this aspect, the present invention provides a method for the provision of optimized process specifications for a cell cultivation process in a reactor system from cultivation data of the cell cultivation process, comprising the steps of:
acquiring cultivation data of the cell cultivation process; and
adapting or generating at least one optimized process specification from acquired cultivation data by applying a Digital Twin obtainable according to the method of the first aspect of the invention.

The process specifications are preferably optimized with respect to one or more process optimization objectives and constraints. The process optimization requires, in particular, a trained RNN, comprising the H matrix, neural network weights W, and biases b. It is performed by solving a non-linear unconstrained optimization problem (e.g. using a stochastic gradient descent algorithm) with the objective to minimize the following loss function:

$$Loss = {\sum }_{K} ( {\sum }_{t} {a}_{K} ( t ) \cdot K ( t ) ) + {w}_{k} \cdot P ( K )$$

where K(t) indicates a process specification at time point t, coefficient α_{K}(t) determines whether the objective is to maximize, to minimize, or to exclude the process specification K at time point t:

$${α}_{K} ( t ) = \{ \begin{matrix}1 & if the objective is to minimize K ( t ) \\ - 1 & if the objective is to maximize K ( t ) \\ 0 & if K ( t ) is not the objective of the optimization\end{matrix}$$

and P(K) is a penalty function of the process specification K, weighted by hyper-parameters wₖ.

In a third aspect, the present invention provides a method for the cultivation of a biological cell in a reactor system. The method comprises the step of: cultivating the biological cell in the reactor system with at least one optimized process specification provided by the method according to the second aspect of the invention. More particularly, according to this aspect, the invention pertains to a method for the provision of optimized process specifications for a cell cultivation process in a reactor system from cultivation data of the cell cultivation process, comprising the steps of:
acquiring cultivation data of the cell cultivation process; and
adapting or generating at least one optimized process specification from acquired cultivation data by applying a Digital Twin obtainable according to the method of the first aspect of the invention.

According to this aspect, the optimized process specifications are used to run a biotechnological production plant. In a preferred embodiment of the invention where e.g. the feeding scheme is optimized, the device (e.g. a computer) controlling the feed pump, will operate according to software which uses the optimized feeding scheme as an input.

Preferably, the process specification is optimized with respect to one or more specifications, selected from: feeding strategy, medium composition, osmolality, medium pH, pO₂ and temperature.

In a forth aspect, the present invention provides a device for the automated control of a biological cell culture in a reactor system. More particularly, according to this aspect, the invention pertains to a device for the automated control of a running biological cell culture process in a reactor system, comprising:
a computing device including a processor, and
a memory, the memory storing program code and the Digital Twin obtainable according to the method of the first aspect of the invention.

According to the invention, the program code, when executed on said processor, causes the computing device to:
acquire cultivation data from the running cell culture in the reactor system, and
adapt or generate process specifications of the reactor system from the acquired cultivation data.

The programmed controller is preferably applied to adapt or optimize process specifications in the running biological cell culture process online. The cell culture process is preferably controlled in a closed loop feedback system wherein the Digital Twin receives real-time information, i.e. cell cultivation data, from online sensors attached to the reactor and from sampling at discrete time points. This sampled information updates the Digital Twin which then consequently leads to a continuous optimization of the process. The online sensors measure e.g. pH, oxygen saturation, biomass concentration, temperature, infrared or Raman spectra. The discrete sampling gives the information about the concentration of compounds, preferably selected from, but not limited to ammonia, glutamine, glucose and lactate, and/or about the product quality, preferably selected from, but not limited to product fragmentation and glycosylation pattern.

According to this aspect, the invention also pertains to a reactor system for the cultivation of a biological cell culture, which comprises said device for the automated control of the biological cell culture and a reactor.

In a further aspect, invention pertains to automated computing means to perform the steps of the invented method for the construction of a Digital Twin of a real biological cell cultivation process according to the first aspect. Accordingly, the invention provides a non-transitory computer-readable storage medium, containing program code for the construction of a Digital Twin for a cell cultivation process, which program code, when executed by a computer, cause the computer to perform the instruction steps of the method of the first aspect.

More particularly, there is provided a non-transitory computer-readable storage medium, containing program code for the construction of a Digital Twin for a cell cultivation process, which program code, when executed by a computer, cause the computer to:
provide dynamic cultivation data from a real cell cultivation process;
provide a mode matrix M of elementary flux modes , extracted from metabolic fluxes of a real biological cell;
reduce the number of and overlaying the elementary flux modes by a trainable matrix H to obtain a reduced matrix \tilde{S}\tilde{M}^{red} of base flux modes;
assign a neural network for describing the kinetics of the individual base flux modes \tilde{S}\tilde{M}^{red};
connect the base flux modes to extracellular reactions of the cell cultivation process;
connect the base flux modes to inflows and outflows to and from the reactor system of the cell cultivation process;
solve the resulting mass balances of substrates, products and biomass; and
train the H matrix and the neural network by the dynamic cultivation data.

According to this further aspect, the invention also provides a computational system for the construction of the Digital Twin. The computational system comprises:
a computing device including a processor, and
a memory, the memory storing instructions for the construction of the Digital Twin, which, when executed by said processor, cause the computing device to cause the computer to perform the instruction steps of the method of the first aspect.

More particularly, there is provided a computational system for the construction of a Digital Twin for a cell cultivation process, the Digital Twin representing a plurality of a biological cell, extracellular reactions and a reactor system, the computational system comprising:
a computing device including a processor, and
a memory, the memory storing instructions for the construction of the Digital Twin, which, when executed by said processor, cause the computing device to:
provide dynamic cultivation data from a real cell cultivation process;
provide a mode matrix M of elementary flux modes, extracted from metabolic fluxes of a real biological cell;
reduce the number of and overlaying the elementary flux modes by a trainable matrix H to obtain a reduced matrix \tilde{S}\tilde{M}^{red} of base flux modes;
assign a neural network for describing the kinetics of the individual base flux modes \tilde{S}\tilde{M}^{red};
connect the base flux modes to extracellular reactions of the cell cultivation process;
connect the base flux modes to inflows and outflows to and from the reactor system of the cell cultivation process;
solve the resulting mass balances of substrates, products and biomass; and
train the H matrix and the neural network by the dynamic cultivation data.

TABLE 1
Formula symbols

| Fommla | | Mathematical | Quantitative | Common |
|---|---|---|---|---|
| Symbol | Meaning and Important Properties | Dimension | Dimension | Unit |
| A | Degradation rate matrix | m × m | 1/time | 1/h |
| αₖ(t) | Determines whether the objective is to maximize, to | 1 | — | — |
| | minimize, or to exclude the process specification K at | | | |
| | time point t | | | |
| bⱼ | A binary variable corresponding to reaction j | 1 | — | — |
| | representing the complexity of the model | | | |
| b_{L} | Biases of the L^{th} layer of the neural network | number of | — | — |
| | | neurons | in the | |
| | | L^{th} layer × 1 | | |
| cᵢ(t) | Concentration of compound i at time point t | 1 | amount of | mol/L |
| | | | substance/ | |
| | | | volume | |
| cᵢ(t) | Intermediate concentration of compound i at time point | 1 | amount of | mol/L |
| | t | | substance / | |
| | | | volume | |
| c_{p,i} ^{measurement} | Measured concentration of compound i, at time point t, | 1 | amount of | mol/L |
| | corresponding to the cultivation run p | | substance/ | |
| | | | volume | |
| c_{p,i}^{measurement} | Measurement standard deviation of the concentration of | 1 | amount of | mol/L |
| | compound i, at time point t, corresponding to the | | substance/ | |
| | cultivation run p | | volume | |
| c_{p,i}^{predicted}(t) | Concentration of compound i, at time point t, | 1 | amount of | mol/L |
| | corresponding to the cultivation run p, as predicted by | | substance/ | |
| | the RNN | volume | | |
| ChL | Average chain length, i.e. the average amount of amino | 1 | — | — |
| | acids combined in a chain of the product | | | |
| comp | Vector of compound names including biomass | m × 1 | — | — |
| comp^{measure} [symbol] | Names of measured compounds including biomass | Num^{comp,measure} [symbol] × 1 | — | — |
| conditions | Conditions that the cultivation process faces, e.g. | Num^{conditions} × 1 | — | — |
| | substrate limitations, phases | | | |
| D(t) | Outflow rate matrix at time point t | m × m | 1/time | 1/h |
| \tilde{D}(t) | Intermediate outflow rate matrix between time point t | m × m | 1/time | 1/h |
| | and t + Δt, calculated with the intermediate cultivation | | | |
| | volume and constant volumetric rates | | | |
| E(t) | System matrix at time point t | m × m | 1/time | 1/h |
| \tilde{E}(t) | Intermediate System matrix between time point t and | m × m | 1/time | 1/h |
| | t + Δt (calculated with \tilde{D}) | | | |
| f(t) | Intracellular fluxes at time point t | 1 × | amount of | mol/ |
| | | Num^{modes,red} | substance/ | (gDW · |
| | | | (biomass in | h) |
| | | | gram Dry | |
| | | | Weight · | |
| | | | time) | |
| F(t) | Inflow rate matrix at time point t | m × m | 1/time | 1/h |
| \tilde{F}(t) | Intermediate Inflow rate matrix between time point t | m × m | 1/time | 1/h |
| | and t + Δt calculated with the intermediate cultivation | | | |
| | volume and constant volumetric rates | | | |
| F_{B}(t) | Volumetric cell bleeding rate at time point t | 1 | Volume/time | L/h |
| F_{F}^{I}(t) | Volumetric feed inflow rate at time point t | 1 | Volume/time | L/h |
| F_{F}^{O}(t) | Volumetric permeate outflow rate at time point t | 1 | Volume/time | L/h |
| fₘₒₙ_{ᵢ} | Monomer factor of the i^{th} monomer mon_i | 1 | — | — |
| F_{S}^{I}(t) | Non-continuous volumetric inflow rate at time point t | 1 | Volume/time | L/h |
| F_{S}^{O}(t) | Non-continuous volumetric outflow rate at time point t | 1 | Volume/time | L/h |
| G(t) | Growth matrix at time point t | m × m | Rate of | Mol/mol |
| | | | compound | biomass |
| | | | production | |
| | | | per existing | |
| | | | Biomass | |
| H | Mode reduction/combination matrix | Num^{modes,red} × | — | — |
| | | Num^{modes} | | |
| h_{u,z} | An element of the H matrix | 1 | — | — |
| K(t) | Process specification (i.e. compound concentrations, | 1 | — | — |
| | reaction rates, feeding amounts, etc.) at time point t | | | |
| k^{deg} | Rate constant of abiotic degradation of Glutamine | 1 | 1/time | 1/h |
| L | Number of hidden layers in the neural network | 1 | — | — |
| Loss | Optimization loss function which needs to be | 1 | — | — |
| | minimized | | | |
| M | Mode matrix | Num^{modes} × n | — | — |
| \tilde{M} | Reduced mode matrix for the measured compounds | Num^{modes} × | — | — |
| | including all modes and all reactions while the | (n − | | |
| | exchange reactions are excluded | Num^{rxns,exchan} [symbol] | | |
| m | Total number of compounds involved in the metabolic | 1 | — | — |
| | network including biomass, m = size (mets) | | | |
| N(t) | Vector of molar amount of compounds at time point t | m × 1 | amount of | mol |
| | in the same order as in the state vector | | substance | |
| ΔN(t) | Amount change of all compounds (including Biomass) | m × 1 | amount of | mol/h |
| | due to feeding between time point t and t + Δt | | substance/ | |
| | | | time | |
| Num^{comp,me} [symbol] | Number of measured compounds, | 1 | — | — |
| | Num^{comp,measured} = size(comp^{measured}) | | | |
| Num^{condition} | Number of conditions that the cell culture faces, e.g. | 1 | — | — |
| | substrate limitations | | | |
| Num^{modes} | Number of elementary flux modes for given flux | 1 | — | — |
| | distributions | | | |
| Num^{modes,re} [symbol] | Numbers of reduced modes | 1 | — | — |
| Num^{rxns,exen} [symbol] | Number of observed reactions, | 1 | — | — |
| | Num^{rxns,excahnge} = size(rxns^{exchange}) | | | |
| Num^{rxns,v}_{^{non}} [symbol] | Number of reactions with nonzero fluxes v_{nonzero} | 1 | — | — |
| n | Total number of reactions involved in the metabolic | 1 | — | — |
| | model, n = size(rxns) | | | |
| p | Index for the current observed cultivation run | 1 | — | — |
| P(K) | Penalty function of the process specification K | 1 | — | — |
| qᵢ | Constant factor for construction of the homogenous | 1 | — | — |
| | system solution. To be calculated from the starting | | | |
| | conditions | | | |
| Qᵢ(t) | Time dependent function for construction of the | — | — | — |
| | particular system solution. To be calculated by | | | |
| | variation of constants | | | |
| rᵢ(t) | Exchange rates of the measured compound i (all | 1 | amount of | mol/ |
| | compounds besides biomass) | | substance/ | (gDW · |
| | | | (biomass in | h) |
| | | | gram Dry | |
| | | | Weight · | |
| | | | time) | |
| rⱼₖ^{est} | Mean of the extracellular rates of measured compounds | 1 | amount of | mol/ |
| | (including biomass) corresponding to reaction j and | | substance/ | (gDW · |
| | condition k, derived by phase search and exchange rate | | (biomass in | h) |
| | estimation | | gram Dry | |
| | | | Weight · | |
| | | | time) | |
| rⱼₖ^{std} | Standard deviations of the extracellular rates of | 1 | amount of | mol/ |
| | measured compounds (including biomass) | | substance/ | (gDW · |
| | corresponding to reaction j and condition k, derived by | | (biomass in | h) |
| | phase search and exchange rate estimation | | gram Dry | |
| | | | Weight · | |
| | | | time) | |
| rxns | Vector of reaction names | n × 1 | — | — |
| rxns^{exchange} | Set of all observed exchange reactions in the model | Num^{rxns,exchange} × | — | — |
| | (corresponding measured | 1 | | |
| | compounds), rxns^{exchange} ⊂ rxns | | | |
| S | Stoichiometric matrix where each entry represents the | m × n | — | — |
| | stoichiometric factor of the corresponding compound i | | | |
| | in the corresponding reaction j | | | |
| sₘₒₙ_{ᵢ} | Denotes the stoichiometric index of the i^{th} monomer | 1 | — | — |
| \tilde{S} | Reduced stoichiometric matrix for the measured | Num^{comp,measure} [symbol] × | — | — |
| | compounds including all reactions while the exchange | (n − | | |
| | reactions are excluded | Num^{rxns,exchan} [symbol] | | |
| \tilde{S}\tilde{M}^{red} | Projected reduced stoichiometric matrix | Num^{modes,red} × | — | — |
| | | Num^{comp,meas} [symbol] | | |
| t | Current time point | 1 | time | h |
| u | Index of rows in H | 1 | — | — |
| valᵢ | i^{th} eigenvalue of \tilde{E} | 1 | — | — |
| vecᵢ | i^{th} eigenvector of \tilde{E} | m × 1 | — | — |
| vⱼ | Flux of reaction j | 1 | — | — |
| vⱼₖ | A metabolic flux denoting the flux of reaction j under | 1 | amount of | mol/ |
| | condition k | | substance/ | (gDW · |
| | | | (biomass in | h) |
| | | | gram Dry | |
| | | | Weight · | |
| | | | time) | |
| vⱼₖ^{est} | Estimated exchange flux from MFA corresponding to | 1 | amount of | mol/ |
| | reaction j and condition k | | substance/ | (gDW · |
| | | | (biomass in | h) |
| | | | gram Dry | |
| | | | Weight · | |
| | | | time) | |
| V(t) | Cultivation volume at time point t | 1 | volume | L |
| V(t) | Intermediate cultivation volume between the time t and | 1 | volume | L |
| | t + Δt | | | |
| ΔV_{F}(t) | Continuous cultivation Volume Change between time | 1 | Volume/ | L/h |
| | point t and t + Δt | | time | |
| ΔV_{S}(t) | Non-continuous cultivation Volume Change between | 1 | Volume/ | L/h |
| | time point t and t + Δt | | time | |
| W_{L} | Matrix of neural network weights between | Num^{layers} [L + | — | — |
| | layer L and L + 1 | 1] × | | |
| | | Num^{layers}[L] | | |
| wₖ | Weighting factors of penalty term P(K) | 1 | — | — |
| x(t) | Biomass concentration in the process at time point t | 1 | concentration | mol/L |
| | | | of | |
| | | | biomass | |
| \tilde{x}(t) | Intermediate Biomass concentration in the process | 1 | concentration | mol/L |
| | between time point t and t + Δt | | of | |
| | | | biomass | |
| X(t) | State vector | m × 1 | concentration | mol/L |
| \tilde{X}(t) | Intermediate state vector between time point t and t + | m × 1 | concentration | mol/L |
| | Δt | | | |
| X^{I}(t) | Inflow concentration state vector | m × 1 | concentration | mol/L |
| \tilde{X}^{I}(t) | Intermediate Inflow concentration state vector between | m × 1 | concentration | mol/L |
| | time point t and t + Δt | | | |
| z | Index of columns in H | 1 | — | — |

[symbol]

indicates data missing or illegible when filed

TABLE 2
Greek letters

| Greek Letter | Meaning | Unit |
|---|---|---|
| Δ | stands for the change or a specific quantity | — |
| λ | penalty factor which weights the model | — |
| | complexity against the description of the | |
| | data and is defined between 0 and 1 | |
| μ(t) | growth rate at time point t | 1/h |
| σ(•) | sigmoidal function (activation function in | — |
| | neural network) | |

### DETAILED DESCRIPTION OF THE FIGURES

FIG. 1 schematically shows the building blocks and structure of the Digital Twin (100) according to the invention. The Digital Twin (100) comprises the reactor model (110), describing all the in- and outlets to the cultivation system, the extracellular reaction model (120), describing all chemical reactions in the cultivation media, and the cell model (130) describing the dynamics of the cells including cellular metabolism and growth. The dynamics of the cell model (130) is obtained by coupling cell metabolism, i.e. metabolic network (131) with a neural network (132).

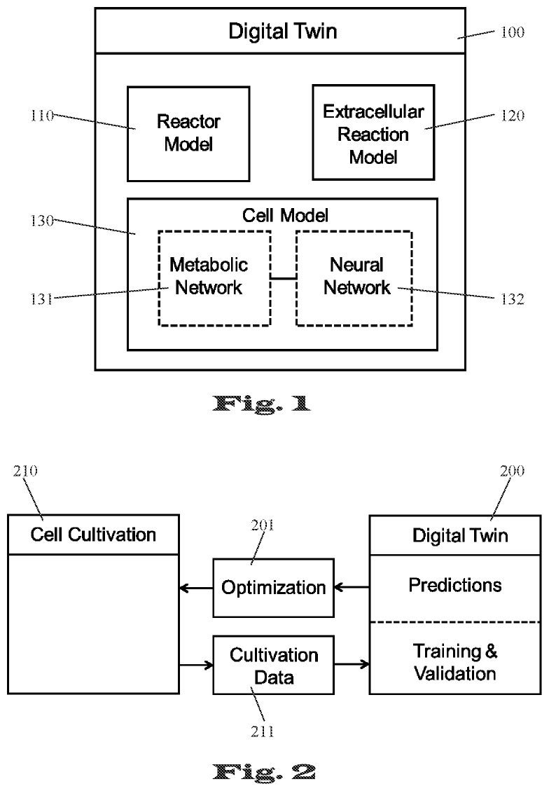

FIG. 2 schematically depicts the Digital Twin (200) in operation mode according to the invention. Cultivation data (211) from the real cell culture (210) are used for the training and validation of the Digital Twin (200). The Digital Twin (200), in turn, is used for predictions aimed at optimizing (201) the cell culture performance (e.g. productivity and growth) and/or the quality of the product produced by the cell culture (210).

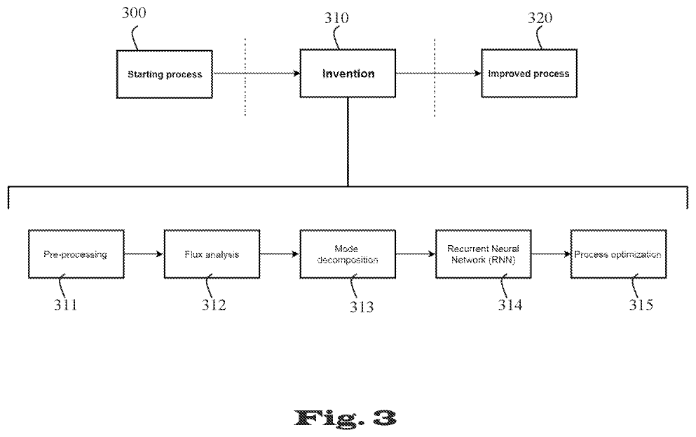

FIG. 3 shows an implemented workflow for a method according to a preferred embodiment of the invention: Starting process specifications (300), i.e. cultivation data, are received and the process specifications (320) are automatically optimized to obtain an improved process. The strategy of the method of the invention (310) is based on a fully automatic and autonomous process which preferably includes initial pre-processing (311) of the cell cultivation data, flux analysis (312) to get the best estimation of the intracellular fluxes therefrom, a mode decomposition (313) of the flux data computed, and the application of a novel recurrent metabolic network model (RNN) (314) which is trained on the basis of the computed flux data. The trained RNN (314) is then applied to an automated process optimization step (315) to obtain the improved process specifications (320).

FIG. 4 shows a flowchart of the process of phase search and exchange rate estimation algorithm (400) according to a preferred embodiment of the invention. The phase search algorithm is a three times nested optimization problem. The linear convex problem solves the estimation of the exchange rates. The global continuous problem finds the optimized positions of the phase borders and the discrete optimization problem estimates the best number of phases. The dashed and dotted lines each indicate the linear convex problem (410), which is nested in the global optimization problem (420), which is nested in the discrete optimization problem (430). Inputs to the phase search and exchange rate estimation are cultivation data as reflected by the cultivation process specification (401) and the time series of (metabolites) concentration measurements (402). The outputs of this module are the estimated extracellular rates (441), i.e. exchange rates, and the detected phase borders (442) of the growth phases of the cultivation process.

FIG. 5 shows a flowchart of the metabolic flux analysis (MFA) algorithm (500) according to a preferred embodiment of the invention: The inputs to the MFA are the estimated extracellular rates (501), obtained from the phase search and exchange rate estimation (see FIG. 4), and the metabolic network (502) of the current cultivation process. The output of the MFA is a set of estimated intracellular metabolic fluxes (510).

FIG. 6 shows a flowchart of the Mode decomposition algorithm (600) according to a preferred embodiment of the present invention: The inputs to the mode decomposition algorithm (600) are the metabolic fluxes (601), derived from MFA (see FIG. 5), and the metabolic network (602) of the current cultivation process. The output is a matrix M (603) of elementary flux modes (EFM); “F_removed” indicates the total number of fluxes remained after removing the elementary fluxes identified at each iteration step of the algorithm.

FIGS. 7A and 7B show a flow chart of the trainable recurrent metabolic network model (RNN) according to a preferred embodiment of the present invention: The RNN contains four distinct parts: the intermediate state model (710), the neural network (720), the flux-based rate estimation (730), and the exponential growth model (740). The panels (700) illustrate the mathematical representation of a single RNN step in detail. The inputs to each RNN step are the compound concentrations and cultivation volume from either the initial status (first step) or from the preceding RNN step. The outputs of each RNN step are the “updated” compound concentrations and cultivation volume. Further inputs to each step of the RNN are the continuous (i.e. feeding-related) cultivation volume change ΔV_{F}(t), the compound amount change due to feeding ΔN(t), and the non-continuous (i.e. sampling-related) cultivation volume change ΔV_{S}(t).

FIG. 8 shows a schematic representation of the matrix multiplication operation according to a preferred embodiment of the present invention which reduces the mode dimensionality in the RNN, corresponding to equation:

\tilde{S}\tilde{M}^{red}=H·[\tilde{S}·\tilde{M}^{T}]^{T}∧H≥0

h_{u,z}≥0∧h_{u,z}∈H

H is a trainable matrix which transforms the number of modes Num^{modes} to a reduced number Num^{modes,red}.

FIG. 9 shows a flowchart of the training and validation of the RNN according to a preferred embodiment of the invention: Inputs are the metabolic network (902), the matrix of elementary flux modes (901), a training set of the cultivation data (905), a subset of the whole cultivation data, and hyper-parameters, such as the number of reduced modes (904), the number of hidden layers or the number of neurons per layer of the neural network. The dashed line indicates the optimization loop (910) for training the RNN. After a successful training, the RNN returns the trained H matrix (907) and the learned weights (W) and biases (b) (906) of the neural network.

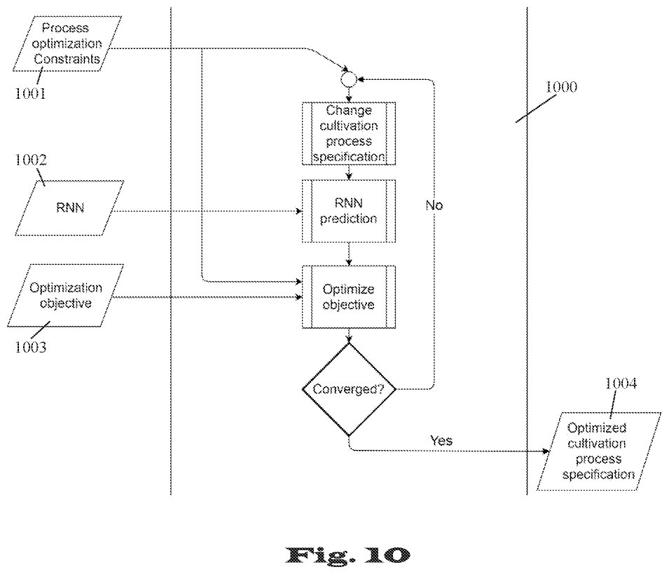

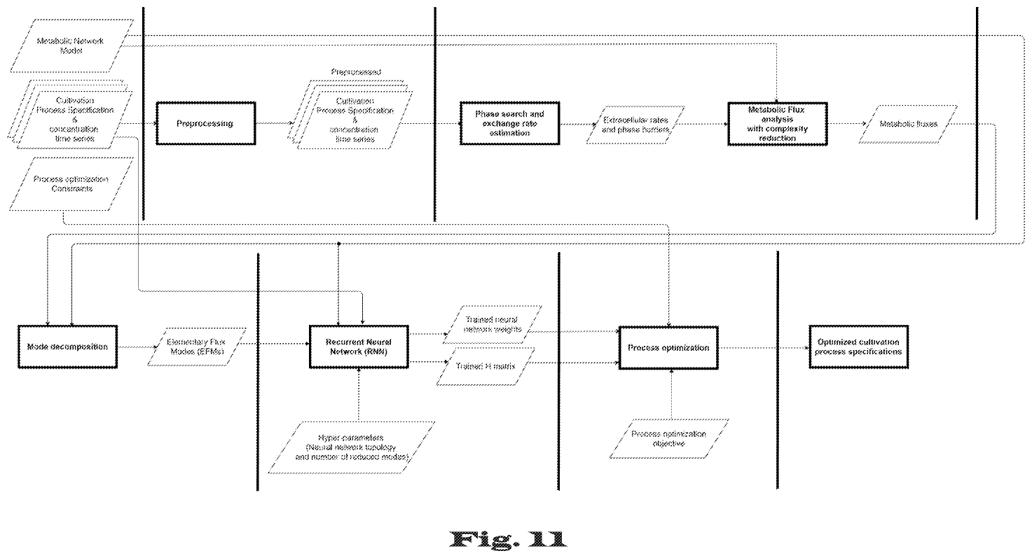

FIG. 10 shows a flowchart of a process optimization algorithm (1000) according to a preferred embodiment of the invention: Inputs to the process optimization are the preset process optimization constraints (1001), the trained recurrent metabolic network (H, W, b) (1002) see FIG. 9, and the one or more optimization objectives (1003) of the intended process optimization. The output is a set of optimized cultivation process specifications (1004).

FIG. 11 shows a flowchart of an overall automated process and all data flows inside the process according to a preferred embodiment of the invention.

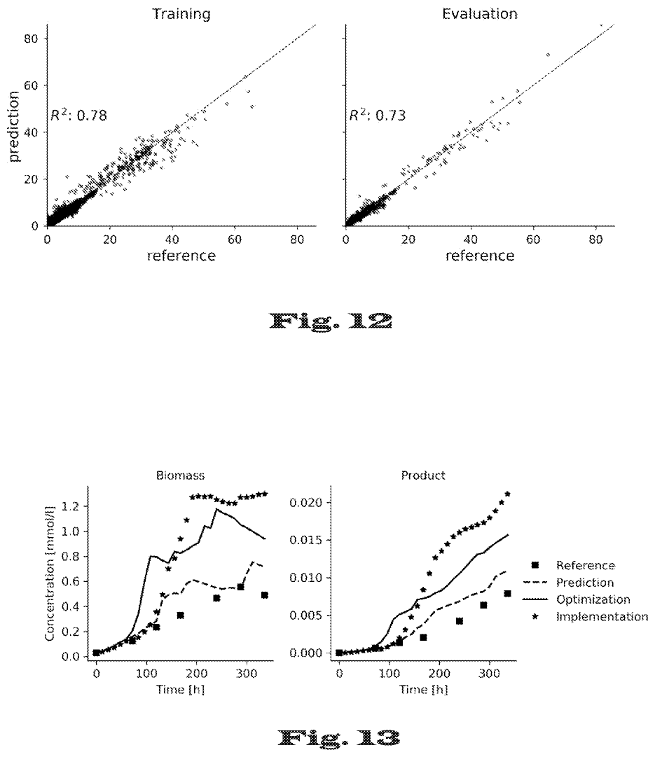

FIG. 12 shows the performance of the model in accordance with the invention on the training (left panel) and evaluation data sets (right panel), respectively. The R² is used to quantify the predictive power of the model. For both panels, x-axis and y-axis indicate the measured and the predicted concentrations, respectively.

FIG. 13 shows graphs of the measured (squares), predicted (dashed line), optimized (solid line), and experimentally implemented (stars) concentrations for biomass (left panel) and the product (right panel) over a single cell culture process in accordance with the invention. In this example the aim for the process optimization was to increase the product titer. The optimized process specifications, provided by an algorithm according to the invention, lead to a higher product titer (compare stars with squares).

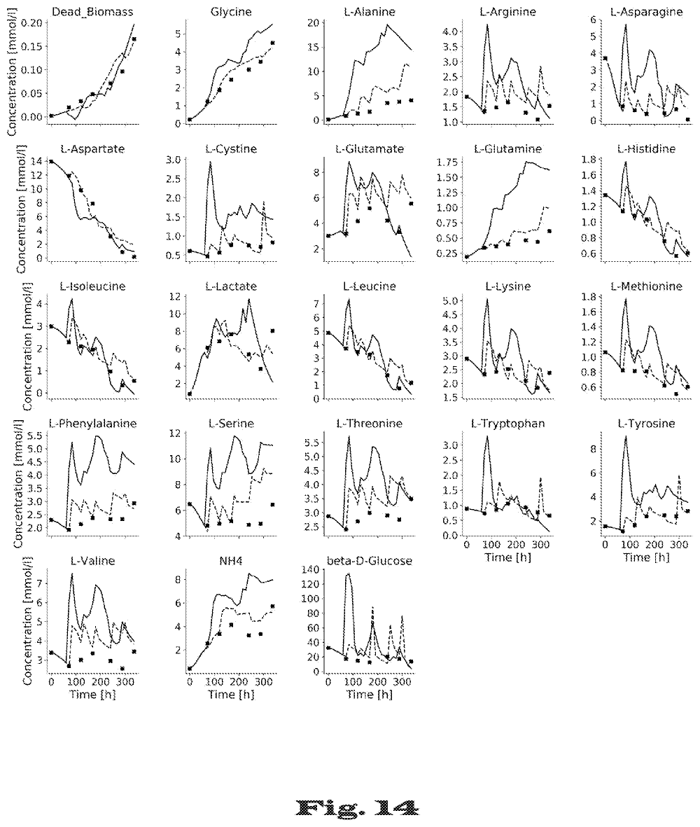

FIG. 14 shows the experimentally measured (squares), the predicted (dashed line), and the optimized (solid line) concentrations for all compounds, besides biomass and product.

### EXAMPLE

In the following, it is demonstrated, how the feeding and the media are optimized in order to increase the final titer by employing the teaching of the present invention.

Experimental setup including reactor setup. An industrial recombinant Chinese Hamster Ovary (CHO) cell line expressing an IgG monoclonal antibody (mAb) through a Glutamine Synthetase (GS) expression system is used in this example. The amino acid composition (in mol-%) of the monoclonal antibody was: Ala 5.4, Arg 3.9, Asn 2.6, Asp 4.3, Cys 4.1, Glu 5.8, Gln 4.2, Gly 3.0, His 4.2, Ile 4.1, Leu 6.3, Lys 4.3, Met 2.2, Phe 3.0, Pro 8.3, Ser 10.2, Thr 5.3, Try 5.5, Tyr 4.2, Val 9.1

During expansion, the cells were cultured in shake flasks and maintained in a humidified incubator at 36° C. and 5% CO2. The cells were passaged every 3-4 days in chemically defined media before seeding at 0.5-1×10⁶ cells/ml into 24 ambr® 15 reactors (Sartorius, Gottingen, Germany). The basal media ActiCHO-P (GE Healthcare), was supplemented with 4 mM L-glutamine and added to the reactor before seeding, so that the starting volume after inoculation was 10 mL. Three feed systems were used being: ActiCHO Feed™-A (feed₁) and ActiCHO Feed™-B (feed₂, GE Healthcare) based on suppliers' information and glucose feed (feed₃) with 2500 mM glucose. The daily feeding volume for feed₁ and feed₂ were 3% and 0.3% of the cell culture volume. Glucose concentration was maintained above 3 g/L by addition of feed₃. 1 mL was sampled on days 3, 5, 7, 10, 12 and 14 for further analyzation.

The cell count, viability and cell diameter were measured by ViCell (Beckman Coulter, Brea, Calif., USA). The Glucose, lactate and ammonia concentrations in the samples were analyzed by a BioProfile Flex analyzer (Nova Biomedical, Waltham, Mass., USA) whereas the amino acids were measured by high-performance liquid chromatography (HP-LC). The titers of the monoclonal Antibody (mAb) were measured by HPLC with a Protein-A column.

Metabolic network. The CHO metabolic network of Hefzi et al. [3] was imported using the software Insilico Discovery™ (Insilico Biotechnology AG, Stuttgart, Germany). The stoichiometric matrix S of the metabolic network was then transferred for further processing to the Digital Twin.

Extracellular network. An extracellular reaction network was not considered in the example.

Training and evaluation. The data set was split into training set (80%) and evaluation set (20%). The Digital Twin learned measured concentrations within the training set. Afterwards, the predictive power of the Digital Twin was evaluated using the evaluation set (see FIG. 12). The neural network f(t) included two hidden layers with 30 and 20 neurons in each layer, respectively, and the number of base flux modes was 10.

Process optimization. The Digital Twin was used to optimize the process. The optimization aimed to increase the product titer experimentally (compare stars with squares in FIG. 13) by adapting the feeding regime and media composition of feed₁ and feed₂. Daily volume additions of each feed were limited between 0 and 1 mL. Feeding was additionally limited by the operation range of the reactor (10-15 mL). Media components were bounded by their solubility limits. Apart from biomass and product, the Digital Twin learned the concentration of all compounds over the process duration (see FIG. 14).

To conclude, in this specific example the aim for the process optimization was to increase the product titer. This example proves that the optimized process specifications, proposed by the invention, lead to a significantly higher product titer (FIG. 13).

### REFERENCES

[1] Antoniewicz, M. R., Kelleher, J. K., & Stephanopoulos, G. (2006). Determination of confidence intervals of metabolic fluxes estimated from stable isotope measurements. Metabolic engineering, 8(4), 324-337.

[2] Chan, S. H. J. & Ji, P. (2011). Decomposing flux distributions into elementary flux modes in genome-scale metabolic networks. Bioinformatics 27,2256-2262.

[3] Hefzi et al. (2016). A Consensus genome-scale reconstruction of Chinese Hamster Ovary (CHO) cell metabolism. Cell Systems, 3(5), 434-44.

1. A method for the construction of a Digital Twin for a cell cultivation process, the Digital Twin representing a plurality of a biological cell, extracellular reactions and a reactor system, the method comprising the steps of:
providing dynamic cultivation data from a real cell cultivation process;
providing a mode matrix M of elementary flux modes, extracted from metabolic fluxes of a real biological cell;
reducing the number of and overlaying the elementary flux modes by a trainable matrix H to obtain a reduced matrix \tilde{S}\tilde{M}^{red} of base flux modes;
assigning a neural network for describing the kinetics of the individual base flux modes \tilde{S}\tilde{M}^{red};
connecting the base flux modes to extracellular reactions of the cell cultivation process;
connecting the base flux modes to inflows and outflows to and from the reactor system of the cell cultivation process;
solving the resulting mass balances of substrates, products and biomass; and
training the H matrix and the neural network by the dynamic cultivation data.

2. The method of claim 1, wherein the projected reduced stoichiometric matrix \tilde{S}\tilde{M}^{red} is derived from metabolic network matrices \tilde{S} and \tilde{M} by applying the trainable positive reduction matrix H to transform the number of modes Num^{modes} to a reduced number Num^{modes,red}:
\tilde{S}\tilde{M}^{red}=H·[\tilde{S}·\tilde{M}^{T}]^{T}
h_{u,z}≥0∧h_{u,z}∈H
wherein the metabolic network matrices \tilde{S} and \tilde{M} are derived from a stoichiometric matrix S of the real biological cell and the mode matrix M, by removing all exchange reactions from both matrices, and including in \tilde{S} only the exchange compounds.

3. The method of claim 1 wherein the mass balances of substrates, products and biomass are solved by a recurrent metabolic network model (RNN), comprising:
an intermediate state model, describing the changes in the cultivation volume and the state vector as a continuous function of time for a certain time step while ensuring correct mass balance;
the neural network, computing the update of the base flux modes f(t) by training the neural network weights W along with their corresponding biases b, where the neurons of the next layer are activated by a sigmoidal activation function σ:
f(t)=σ(W_{L}·σ(W_{L-1}· . . . ·σ(W₀·\tilde{X}(t)+b₀)+ . . . +b_{L-1})+b_{L})
L denotes the index of the last hidden layer;
a flux-based rate estimation obtaining the extracellular rates by:
[μ(t), r(t)]=f(t)·\tilde{S}\tilde{M}^{red}=f(t)·H·[\tilde{S}·\tilde{M}^{T}]^{T}
h_{u,z}≥0∧h_{u,z}∈H
and
an exponential growth model calculating the state vector for the next time step t+Δt.

4. The method according to claim 3, wherein the training the RNN is performed by using a first subset of the cultivation data (training set), by minimizing the loss function:
$$Loss = {\sum }_{p , t , i} {( \frac{{c}_{p , i}^{predicted} ( t ) - {c}_{p , i}^{m e a s u r e m e n t} ( t )}{{c}_{p , i}^{measurement , std} ( t )} )}^{2}$$
where i is an indication of compounds including biomass, c_{p,i}^{measurement}(t) is the measured concentration of compound i, c_{p,i}^{measurement,std}(t) is the measurement standard deviation of the concentration of compound i, c_{p,i}^{predicted}(t) is the predicted concentration of compound i, each at time point t and each corresponding to the selected cultivation run p.

5. The method according to claim 4, further comprising the step of:
evaluating the trained RNN by calculating Loss on the basis of a second subset of the cultivation data (evaluation set), the second subset being different from the first subset.

6. The method according to claim 1, wherein the mode matrix M of elementary flux modes is obtained by mode decomposition, the method comprising the steps of:
transforming all metabolic fluxes to separate off reversible reactions to obtain all irreversible reactions;
minimizing an objective function and deactivation of inactivate transformers are recurrently applied to obtain elementary flux modes, the objective function being:
min(Num^{rxns,v}^{_{nonzero}}⁾
where Num^{rxns,v}^{_{nonzero}} is the number of reactions with non-zero fluxes; and
collecting all elementary flux modes identified and stacking them into a mode matrix M.

7. A method for the provision of optimized process specifications for a cell cultivation process in a reactor system from cultivation data of the cell cultivation process, comprising the steps of:
acquiring cultivation data of the cell cultivation process; and
adapting or generating at least one optimized process specification from acquired cultivation data by applying a Digital Twin obtainable according to claim 1.

8. A method for the cultivation of biological cells in a reactor system, comprising the steps of:
cultivating the biological cells in the reactor system;
acquiring cultivation data from the cell culture in the reactor system;
adapting or generating at least one optimized process specification from the acquired cultivation data by applying a Digital Twin obtainable according to claim 1; and
applying the at least one optimized process specification to the reactor system.

9. The method of claim 8, wherein the process specification is optimized with respect to one or more specifications, selected from: feeding strategy, medium composition, osmolality, medium pH, pO₂ and temperature.

10. A device for the automated control of a running biological cell culture in a reactor system, comprising:
a computing device including a processor, and
a memory, the memory storing program code and the Digital Twin obtainable according to claim 1, which, when executed on the processor, cause the computing device to:
acquire cultivation data from the running cell culture in the reactor system, and
adapt or generate process specifications of the reactor system from the acquired cultivation data.

11. A reactor system for the cultivation of a biological cell culture, comprising the device of claim 10 and a reactor.

12. A non-transitory computer-readable storage medium, containing program code for the construction of a Digital Twin for a cell cultivation process, the Digital Twin representing a plurality of a biological cell, extracellular reactions and a reactor system, which program code, when executed by a computer, cause the computer to:
provide dynamic cultivation data from a real cell cultivation process;
provide a mode matrix M of elementary flux modes, extracted from metabolic fluxes of a real biological cell;
reduce the number of and overlaying the elementary flux modes by a trainable matrix H to obtain a reduced matrix \tilde{S}\tilde{M}^{red} of base flux modes;
assign a neural network for describing the kinetics of the individual base flux modes \tilde{S}\tilde{M}^{red};
connect the base flux modes to extracellular reactions of the cell cultivation process;
connect the base flux modes to inflows and outflows to and from the reactor system of the cell cultivation process;
solve the resulting mass balances of substrates, products and biomass; and
train the H matrix and the neural network by the dynamic cultivation data.

13. The non-transitory computer-readable storage medium according to claim 12, wherein the projected reduced stoichiometric matrix \tilde{S}\tilde{M}^{red} is derived from metabolic network matrices \tilde{S} and \tilde{M} by applying the trainable positive reduction matrix H to transform the number of modes Num^{modes} to a reduced number Num^{modes,red};
\tilde{S}\tilde{M}^{red}=H·[\tilde{S}·\tilde{M}^{T}]^{T}
h_{u,z}≥0∧h_{u,z}∈H
wherein the metabolic network matrices \tilde{S} and \tilde{M} are derived from a stoichiometric matrix S of the real biological cell and the mode matrix M, by removing all exchange reactions from both matrices, and including in \tilde{S} only the exchange compounds.

14. A computational system for the construction of a Digital Twin for a cell cultivation process, the Digital Twin representing a plurality of a biological cell, extracellular reactions and a reactor system, the computational system comprising:
a computing device including a processor, and
a memory, the memory storing instructions for the construction of the Digital Twin, which, when executed by said processor, cause the computing device to:
provide dynamic cultivation data from a real cell cultivation process;
provide a mode matrix M of elementary flux modes, extracted from metabolic fluxes of a real biological cell;
reduce the number of and overlaying the elementary flux modes by a trainable matrix H to obtain a reduced matrix \tilde{S}\tilde{M}^{red} of base flux modes;
assign a neural network for describing the kinetics of the individual base flux modes \tilde{S}\tilde{M}^{red}.
connect the base flux modes to extracellular reactions of the cell cultivation process;
connect the base flux modes to inflows and outflows to and from the reactor system of the cell cultivation process;
solve the resulting mass balances of substrates, products and biomass; and
train the H matrix and the neural network by the dynamic cultivation data.

15. The computational system according to claim 14, wherein the projected reduced stoichiometric matrix \tilde{S}\tilde{M}^{red} is derived from metabolic network matrices \tilde{S} and \tilde{M} by applying the trainable positive reduction matrix H to transform the number of modes Num^{modes} to a reduced number Num^{modes,red}:
\tilde{S}\tilde{M}^{red}=H·[\tilde{S}·\tilde{M}^{T}]^{T}
h_{u,z}≥0∧h_{u,z}∈H
wherein the metabolic network matrices \tilde{S} and \tilde{M} are derived from a stoichiometric matrix S of the real biological cell and the mode matrix M, by removing all exchange reactions from both matrices, and including in \tilde{S} only the exchange compounds.

16. The non-transitory computer-readable storage medium according to claim 12, wherein the mass balances of substrates, products and biomass are solved by a recurrent metabolic network model (RNN), comprising:
an intermediate state model, describing the changes in the cultivation volume and the state vector as a continuous function of time for a certain time step while ensuring correct mass balance;
the neural network, computing the update of the base flux modes f(t) by training the neural network weights W along with their corresponding biases b, where the neurons of the next layer are activated by a sigmoidal activation function σ:
f(t)=σ(W_{L}·σ(W_{L-1}· . . . ·σ(W₀·\tilde{X}(t)+b₀)+ . . . +b_{L-1})+b_{L})
L denotes the index of the last hidden layer;
a flux-based rate estimation obtaining the extracellular rates by:
[μ(t), r(t)]=f(t)·\tilde{S}\tilde{M}^{red}=f(t)·H·[\tilde{S}·\tilde{M}^{T}]^{T}
h_{u,z}≥0∧h_{u,z}∈H
and
an exponential growth model calculating the state vector for the next time step t+Δt.

17. The non-transitory computer-readable storage medium according to claim 16, wherein the training the RNN is performed by using a first subset of the cultivation data (training set), by minimizing the loss function:
$$Loss = {\sum }_{p , t , i} {( \frac{{c}_{p , i}^{predicted} ( t ) - {c}_{p , i}^{m e a s u r e m e n t} ( t )}{{c}_{p , i}^{measurement , std} ( t )} )}^{2}$$
where i is an indication of compounds including biomass, c_{p,i}^{measurement}(t) is the measured concentration of compound i, c_{p,i}^{measurement,std}(t) is the measurement standard deviation of the concentration of compound i, c_{p,i}^{predicted}(t) is the predicted concentration of compound i, each at time point t and each corresponding to the selected cultivation run p.

18. The non-transitory computer-readable storage medium according to claim 17, further comprising the step of:
evaluating the trained RNN by calculating Loss on the basis of a second subset of the cultivation data (evaluation set), the second subset being different from the first subset.

19. The non-transitory computer-readable storage medium according to claim 12, wherein the mode matrix M of elementary flux modes is obtained by mode decomposition, the method comprising the steps of:
transforming all metabolic fluxes to separate off reversible reactions to obtain all irreversible reactions;
minimizing an objective function and deactivation of inactivate transformers are recurrently applied to obtain elementary flux modes, the objective function being:
min(Num^{rxns,v}^{_{nonzero}}⁾
where Num^{rxns,v}^{_{nonzero}} is the number of reactions with non-zero fluxes; and
collecting all elementary flux modes identified and stacking them into a mode matrix M.

20. The computational system according to claim 14, wherein the mass balances of substrates, products and biomass are solved by a recurrent metabolic network model (RNN), comprising:
an intermediate state model, describing the changes in the cultivation volume and the state vector as a continuous function of time for a certain time step while ensuring correct mass balance;
the neural network, computing the update of the base flux modes f(t) by training the neural network weights W along with their corresponding biases b, where the neurons of the next layer are activated by a sigmoidal activation function σ:
f(t)=σ(W_{L}·σ(W_{L-1}· . . . ·σ(W₀·\tilde{X}(t)+b₀)+ . . . +b_{L-1})+b_{L})
L denotes the index of the last hidden layer;
a flux-based rate estimation obtaining the extracellular rates by:
[μ(t), r(t)]=f(t)·\tilde{S}\tilde{M}^{red}=f(t)·H·[S·M^{T}]^{T}
h_{u,z}≥0∧h_{u,z}∈H
and
an exponential growth model calculating the state vector for the next time step t+Δt.

21. The computational system according to claim 20, wherein the training the RNN is performed by using a first subset of the cultivation data (training set), by minimizing the loss function:
$$Loss = {\sum }_{p , t , i} {( \frac{{c}_{p , i}^{predicted} ( t ) - {c}_{p , i}^{m e a s u r e m e n t} ( t )}{{c}_{p , i}^{measurement , std} ( t )} )}^{2}$$
where i is an indication of compounds including biomass, c_{p,i}^{measurement}(t) is the measured concentration of compound i, c_{p,i}^{measurement,std}(t) is the measurement standard deviation of the concentration of compound i, c_{p,i}^{predicted}(t) is the predicted concentration of compound i, each at time point t and each corresponding to the selected cultivation run p.

22. The computational system according to claim 21, further comprising the step of:
evaluating the trained RNN by calculating Loss on the basis of a second subset of the cultivation data (evaluation set), the second subset being different from the first subset.

23. The computational system according to claim 14, wherein the mode matrix M of elementary flux modes is obtained by mode decomposition, the method comprising the steps of:
transforming all metabolic fluxes to separate off reversible reactions to obtain all irreversible reactions;
minimizing an objective function and deactivation of inactivate transformers are recurrently applied to obtain elementary flux modes, the objective function being:
min(Num^{rxns,v}^{_{nonzero}}⁾
where Num^{rxns,v}^{_{nonzero}} is the number of reactions with non-zero fluxes; and
collecting all elementary flux modes identified and stacking them into a mode matrix M.
